(19) United States
(12) Patent Application Publication
(10) Pub. No.: US 2023/0093984 A1
(43) Pub. Date: Mar. 30, 2023

(54) PRECISION GRAPHENE NANORIBBON WIRES FOR MOLECULAR ELECTRONICS SENSING AND SWITCHING DEVICES

(71) Applicant: The Regents of the University of California, Oakland, CA (US)

(72) Inventors: Felix Fischer, Oakland, CA (US); Ryan McCurdy, Hayward, CA (US)

(21) Appl. No.: 17/932,520

(22) Filed: Sep. 15, 2022

Related U.S. Application Data
(60) Provisional application No. 63/261,255, filed on Sep. 15, 2021.

(51) Int. Cl. C12Q 1/6869; C01B 32/194; G01N 27/414

(52) U.S. Cl. CPC C12Q 1/6869; C01B 32/194; G01N 27/4148; G01N 27/4145; G01N 27/4146; C01B 2204/065; B82Y 40/00

(57) ABSTRACT

A precision graphene nanoribbon (GNR) bridge molecule can include: a central GNR having a precision structure selected the following structural types: armchair, zigzag, cove, chevron, and fjord; a functional anchoring group at either end of the GNR selected from the following: amine, thiol, thioether, stannane, halide, boronic acid, boronic ester, azide, and carbene; a central functional conjugation group at a precisely specified location; and edge group functionalization with solubilizing groups selected from the following: linear and branched alkyl chains, substituted aromatic rings, oligoethylene glycol, carboxylic acids, and sulfonic acids.

### CROSS-REFERENCE TO RELATED APPLICATION

The present application claims priority to U.S. Provisional Application No. 63/261,255 entitled “PRECISION GRAPHENE NANORIBBON WIRES FOR MOLECULAR ELECTRONICS SENSING AND SWITCHING DEVICES”, and filed on Sep. 15, 2021. The entire contents of the above-listed application is hereby incorporated by reference for all purposes.

### GOVERNMENT RIGHTS

This invention was made with Government support under Grant No. N00014-16-1-2921, and under Grant No. N00014-19-1-2503 awarded by the Department of Defense. The Government has certain rights in the invention.

### TECHNICAL FIELD

The present disclosure relates generally to the synthesis and the manufacture of molecular electronic devices, and more particularly to sensors, switches, and CMOS chip-based integrated circuits.

### BACKGROUND AND SUMMARY

The term molecular electronics as used herein generally refers to molecules that have been integrated into electronic circuit architectures as functional elements.

The term graphene nanoribbons (GNRs) as used herein generally refers to electrically conducting (semiconducting or metallic) atomically thin macromolecules structurally derived from the lattice of two-dimensional (2D) graphene.

Certain embodiments of the disclosed technology include a method to produce and use GNRs as molecular wires for GNR-based integrated circuits, the method comprising any or all of the following: the design/engineering/synthesis of GNRs featuring precise length, width, and edge structure; the design/engineering/synthesis of GNRs featuring a semiconducting or metallic band structure; the design/engineering/synthesis of GNRs featuring solubilizing side chains to allow for processing in organic solvents, water, aqueous buffered solutions, and organic melts; the design/engineering/synthesis of GNRs featuring a central functional core (e.g. one or more molecular recognition domains, one or more electron spin system, one or more quantum dots, one or more qubits, one or more heterojunction interfaces, one or more symmetry protected topological states); the design/engineering/synthesis of GNRs featuring functional groups as anchoring points at either end of the ribbon that can form covalent or coordinative bonds to metal electrodes; the design/engineering/synthesis of highly conductive GNRs; the design/engineering/synthesis of GNRs as highly sensitive wires for sensors and switches; the design/engineering/synthesis of GNRs for nano-electrode based molecular electronics circuits; and the design/engineering/synthesis of GNRs in a form that can be deployed on a CMOS chip for efficient mass manufacturing of such devices.

Certain embodiments may include means of fabricating such ribbons, and such CMOS chips with such ribbons as functional circuit elements.

Certain embodiments may include applications and methods of use for such GNR molecular electronic devices for areas such as DNA sequencing, detection of diverse analytes, or electronic switching in circuits for logic or communications applications.

### BRIEF DESCRIPTION OF THE DRAWINGS

The present disclosure will be better understood from reading the following description of non-limiting embodiments, with reference to the attached drawings, wherein below:

FIG. 1 illustrates a functional 9-AGNR with a discrete length in accordance with certain embodiments of the present disclosure;

FIG. 2 illustrates a functional 9-AGNR with a discrete length in accordance with certain embodiments of the present disclosure;

FIG. 3 illustrates an example of the bottom-up synthesis of a possible embodiment of a protected molecular building block for a 9-AGNR;

FIG. 4 illustrates an example of the bottom-up synthesis of a key intermediate in the synthesis of a molecular building block featuring solubilizing groups compatible with organic solvents;

FIG. 5 illustrates an example of the bottom-up synthesis of a key intermediate in the synthesis of molecular building block representing a functional core;

FIG. 6 illustrates an example of the bottom-up synthesis of a protected thiol-based anchoring group;

FIG. 7 illustrates an example of the functional integration of a protected thiol-based anchoring group along with the iterative cross-coupling homologation sequence that extends the length of the GNR polymer precursor by one monomer building block at a time;

FIG. 8 illustrates an example of the final assembly of the left/right and right wing with the functional core to give a possible embodiment of a fully functionalized precision 9-AGNR; and

FIG. 9 illustrates certain embodiments of a GNR based sensor for DNA/RNA sequencing (top) and the possible embodiment of a GNR based electrical or magnetic switch for logic operations and computing.

### DETAILED DESCRIPTION

Bottom-up synthesized graphene nanoribbons (GNRs) have emerged as one of the most promising materials for post-silicon integrated circuit architectures and have already demonstrated the ability to overcome many of the grand challenges encountered by devices based on carbon nanotubes or photolithographically patterned graphene. GNR electronic devices give birth to a new field of synthetic electronics that could enable the next generation of electronic circuits and sensors. Embodiments of the present disclosure may overcome one of the grand challenges in graphene nanoelectronics technology: the controlled assembly of highly parallelized GNR electronic device architectures. Embodiments of the disclosure may provide for a highly scalable multiplexed approach that increases the density of GNR-based transistors, used herein for single molecule biomolecular sensing, electrical switching, magnetic switching, and logic operations form a single device/chip scale to 16,000 to >1,000,000 parallel transistors on a single integrated circuit, for example. Embodiments of the present disclosure may represent a technological advancement that overcomes a formidable engineering challenge and gives access to GNR-based functional materials and electronic devices that can be seamlessly integrated with, augment, and even outperform current semiconductor nanotechnology.

Embodiments of the present disclosure may provide for the design, engineering, and bottom-up synthesis of precise graphene nanoribbons (GNRs) comprising any or all of the following: a plurality of semiconducting or metallic GNRs featuring a variety of lengths L (where L=10, 20, 30, 40, 50, 100 or more fundamental repeat units of the GNR monomer building block), widths N (where N=5, 6, 7, 8, 9, 10, 11, 12, 13, 14, 15, 16, 17, 18, 19, 20 is the number of carbon atoms counted in a line across the width of the ribbon), and edge structures (armchair, zigzag, cove, chevron, fjord, or any combination thereof).

Embodiments of the present disclosure may provide for the design, the engineering, and the bottom-up synthesis of precise graphene nanoribbons (GNRs) wherein the GNRs comprise a plurality of functional solubilizing groups along the edges of the ribbon.

Embodiments of the present disclosure may provide for the design, engineering, and bottom-up synthesis of precise graphene nanoribbons (GNRs) wherein the GNRs comprise a plurality of functional anchoring groups at either end of the ribbon that covalently or coordinatively form an electrical connection to a plurality of metal electrodes.

In certain embodiments, a GNR of the disclosure comprises some or all of the above characteristics along with a central functional core featuring a plurality of molecular recognition domains. These molecular recognition domains typically interact with biologically relevant targets through covalent or non-covalent interactions.

In certain embodiments, a GNR of the disclosure comprises some or all of the above characteristics along with a central functional core featuring a precise sequence of a plurality of heterojunction interfaces, a plurality of electron spin system, a plurality of quantum dots, a plurality of qubits, and/or a plurality of symmetry protected topological states.

In certain embodiments, GNRs of the present disclosure feature the following structure, wherein n and m are integers >2:

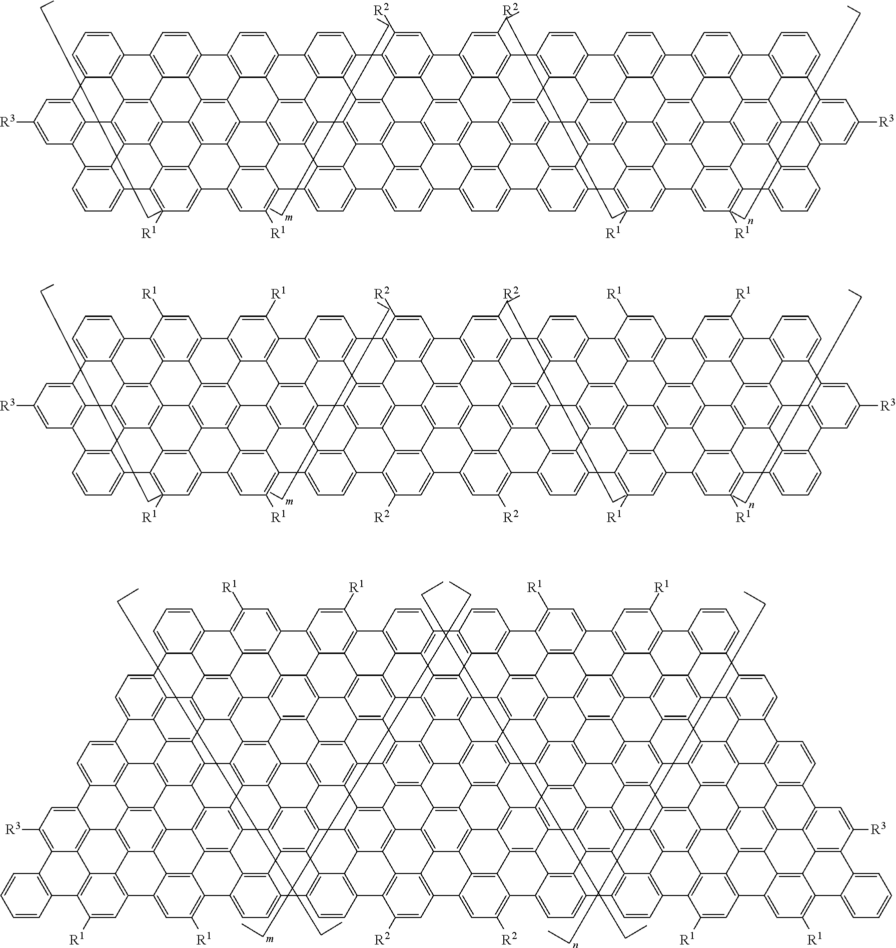

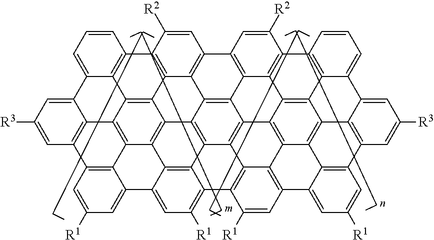

In further embodiments R¹ may be each individually selected from: —H, linear and branched —CₙH₂ₙ₊₁ wherein n=4, 5, 6, 7, 8, 9, 10, 11, 12, 14, 15, 16, 17, 18, 19, 20; —O((CH₂CH₂)O)ₙCH₃ wherein n=2, 3, 4, 5, 6; —CₙH₂ₙCOOH wherein n=3, 4, 5, 6, 7, 8, 9, 10, 11, 12, 14, 15, 16, 17, 18, 19;

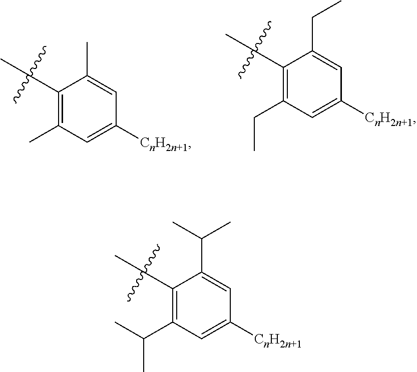

with linear and branched —CₙH₂ₙ₊₁ wherein n=4, 5, 6, 7, 8, 9, 10, 11, 12; and/or

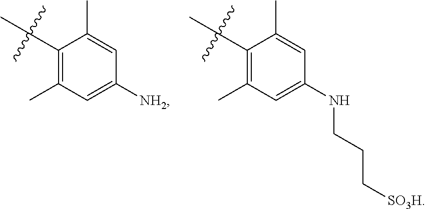

In further embodiments R² may be each individually selected from: —H, linear and branched —CₙH₂ₙNH₂ wherein n=4, 5, 6, 7, 8, 9, 10, 11, 12, 14, 15, 16, 17, 18, 19, 20; —(O(CH₂CH₂))ₙOH wherein n=2, 3, 4, 5, 6; —(O(CH₂CH₂))ₙNH₂ wherein n=2, 3, 4, 5, 6; —CₙH₂ₙCOOH wherein n=3, 4, 5, 6, 7, 8, 9, 10, 11, 12, 14, 15, 16, 17, 18, 19; and/or

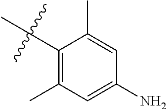 [SMILES: *c1c(C)cc(N)cc1C]

In further embodiments R³ may be each individually selected from: —H, —NH₂, —SH, —S—CₙH₂ₙ₊₁ wherein n=1-6, —OH, —Sn(CH₃)₃, Sn(C₄H₉)₃, —F, —Cl, —Br, —I, —B(OH)₂, —B(OCH₃)₂, —B(OiPr)₂, —B(pin), B(MIDA), -azide, -carbene. In certain embodiments R³ may be each individually selected from a heterocyclic ring fused to the GNR edge selected from the following:

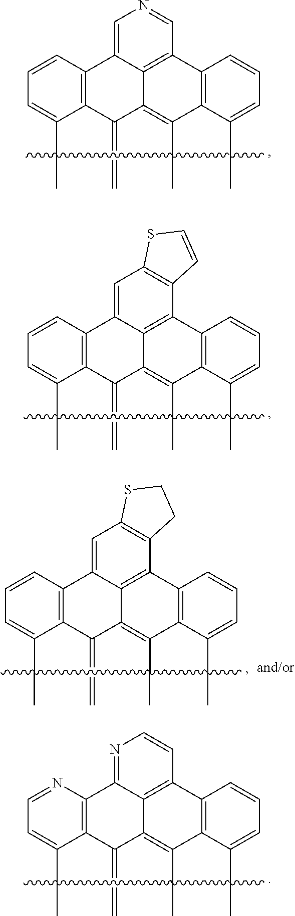

In certain embodiments, GNRs of the disclosure may feature a structure of the following:

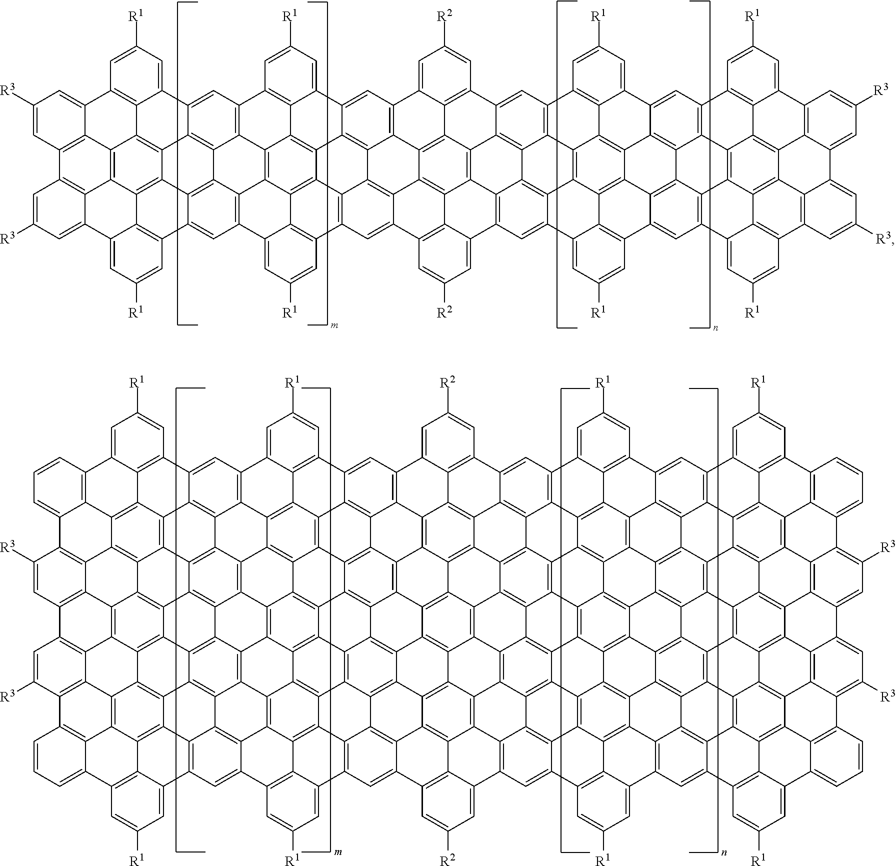

wherein n and m are integers >2.

In further embodiments R¹ may be each individually selected from the following: —H, linear and branched —CₙH₂ₙ₊₁ wherein n=4, 5, 6, 7, 8, 9, 10, 11, 12, 14, 15, 16, 17, 18, 19, 20; —O((CH₂CH₂)O)ₙCH₃ wherein n=2, 3, 4, 5, 6; —CₙH₂ₙCOOH wherein n=3, 4, 5, 6, 7, 8, 9, 10, 11, 12, 14, 15, 16, 17, 18, 19;

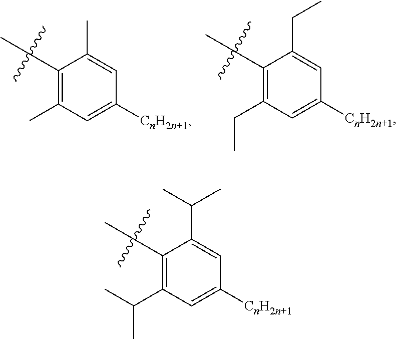

with linear and branched —CₙH₂ₙ₊₁ wherein n=4, 5, 6, 7, 8, 9, 10, 11, 12; and/or

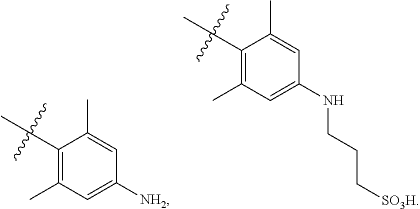

In further embodiments R² may be each individually selected from the following: —H, linear and branched —CₙH₂ₙNH₂ wherein n=4, 5, 6, 7, 8, 9, 10, 11, 12, 14, 15, 16, 17, 18, 19, 20; —(O(CH₂CH₂))ₙOH wherein n=2, 3, 4, 5, 6; —(O(CH₂CH₂))ₙNH₂ wherein n=2, 3, 4, 5, 6; —CₙH₂ₙCOOH wherein n=3, 4, 5, 6, 7, 8, 9, 10, 11, 12, 14, 15, 16, 17, 18, 19; and/or

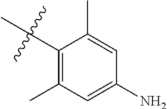 [SMILES: *c1c(C)cc(N)cc1C]

In further embodiments R³ may be each individually selected from the following: —H, —NH₂, —SH, —S—CₙH₂ₙ₊₁ wherein n=1-6, —OH, —Sn(CH₃)₃, Sn(C₄H₉)₃, —F, —Cl, —Br, —I, —B(OH)₂, —B(OCH₃)₂, —B(OiPr)₂, —B(pin), B(MIDA), -azide, -carbene. In certain embodiments R³ may be each individually selected from a heterocyclic ring fused to the GNR edge selected from the following:

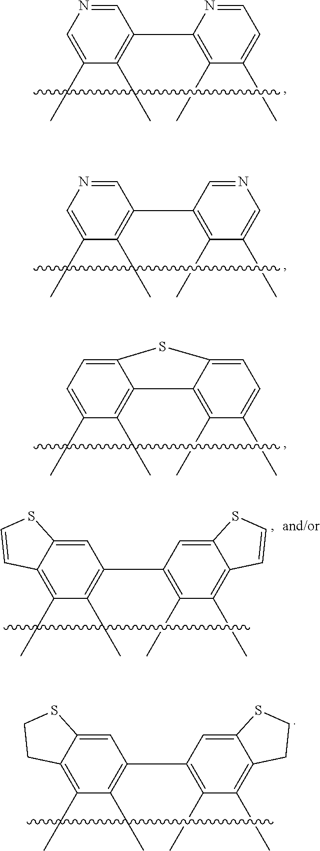

In certain embodiments, GNRs of the present disclosure may feature a structure of the following:

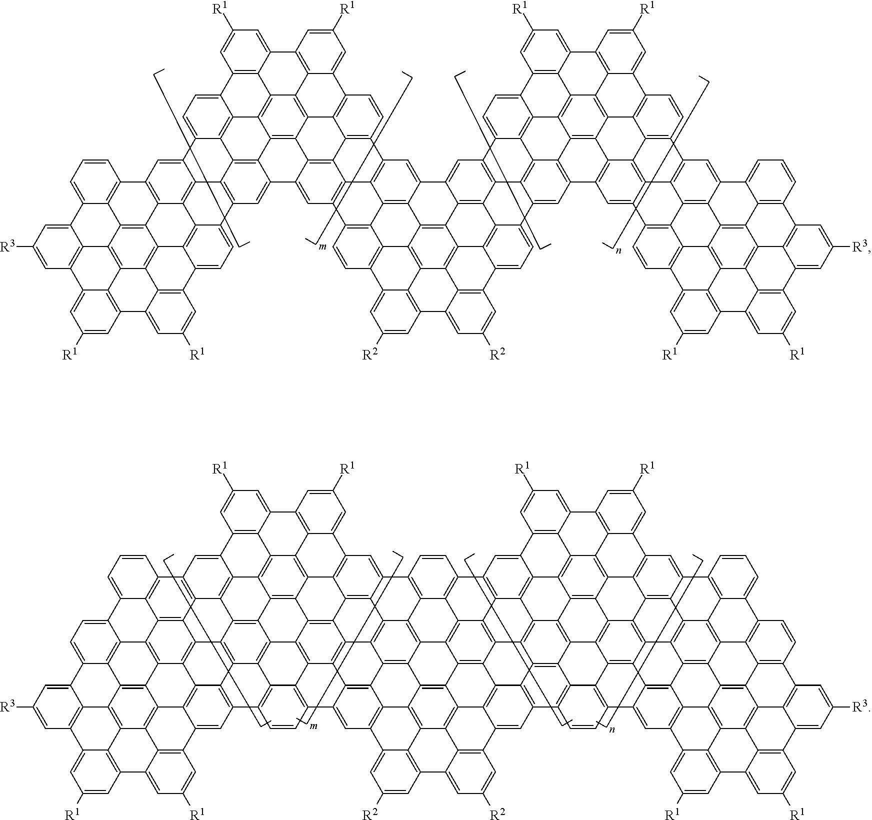

wherein n and m are integers >2.

In further embodiments R¹ may be each individually selected from the following: —H, linear and branched —CₙH₂ₙ₊₁ wherein n=4, 5, 6, 7, 8, 9, 10, 11, 12, 14, 15, 16, 17, 18, 19, 20; —O((CH₂CH₂)O)ₙCH₃ wherein n=2, 3, 4, 5, 6; —CₙH₂ₙCOOH wherein n=3, 4, 5, 6, 7, 8, 9, 10, 11, 12, 14, 15, 16, 17, 18, 19;

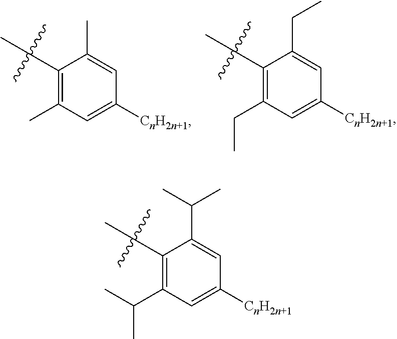

with linear and branched —CₙH₂ₙ₊₁ wherein n=4, 5, 6, 7, 8, 9, 10, 11, 12; and/or

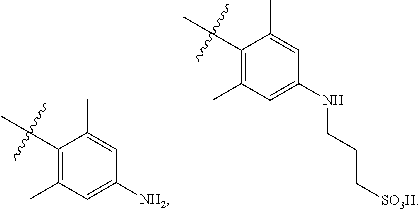

In further embodiments R² may be each individually selected from the following: —H, linear and branched —CₙH₂ₙNH₂ wherein n=4, 5, 6, 7, 8, 9, 10, 11, 12, 14, 15, 16, 17, 18, 19, 20; —(O(CH₂CH₂))ₙOH wherein n=2, 3, 4, 5, 6; —(O(CH₂CH₂))ₙNH₂ wherein n=2, 3, 4, 5, 6; —CₙH₂ₙCOOH wherein n=3, 4, 5, 6, 7, 8, 9, 10, 11, 12, 14, 15, 16, 17, 18, 19; and/or

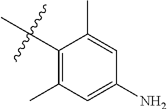 [SMILES: *c1c(C)cc(N)cc1C]

In further embodiments R³ may be each individually selected from the following: —H, —NH₂, —SH, —S—CₙH₂ₙ₊₁ wherein n=1-6, —OH, —Sn(CH₃)₃, Sn(C₄H₉)₃, —F, —Cl, —Br, —I, —B(OH)₂, —B(OCH₃)₂, —B(OiPr)₂, —B(pin), B(MIDA), -azide, -carbene. In certain embodiments R³ may be each individually selected from a heterocyclic ring fused to the GNR edge selected from the following:

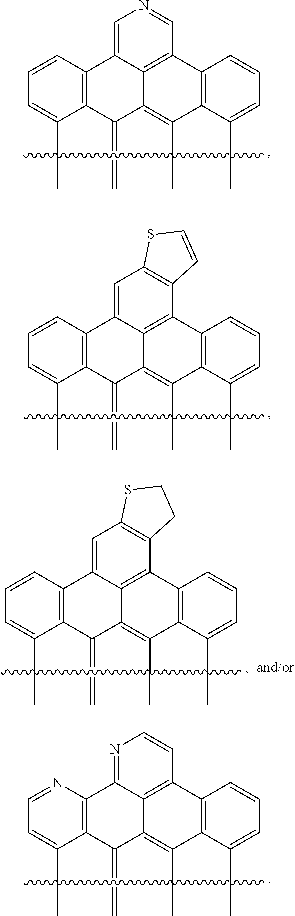

In certain embodiments, GNRs of the present disclosure may feature a structure of the following:

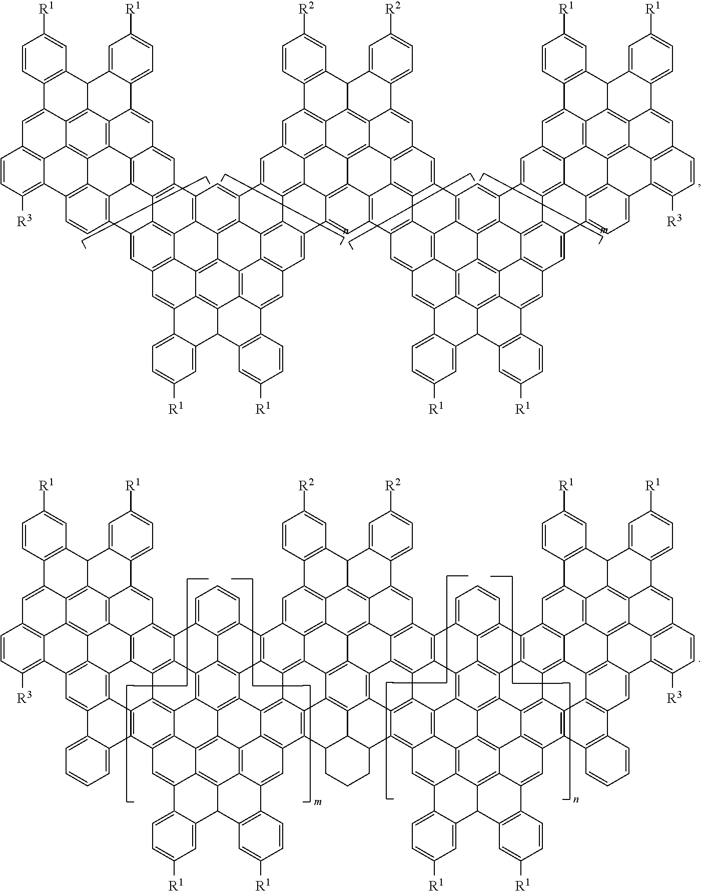

wherein n and m are integers >2.

In further embodiments R¹ may be each individually selected from the following: —H, linear and branched —CₙH₂ₙ₊₁ wherein n=4, 5, 6, 7, 8, 9, 10, 11, 12, 14, 15, 16, 17, 18, 19, 20; —O((CH₂CH₂)O)ₙCH₃ wherein n=2, 3, 4, 5, 6; —CₙH₂ₙCOOH wherein n=3, 4, 5, 6, 7, 8, 9, 10, 11, 12, 14, 15, 16, 17, 18, 19;

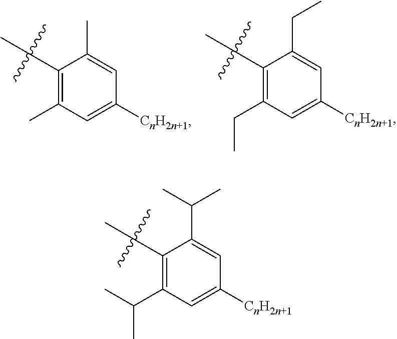

with linear and branched —CₙH₂ₙ₊₁ wherein n=4, 5, 6, 7, 8, 9, 10, 11, 12; and/or

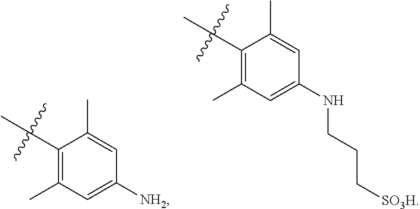

In further embodiments R² may be each individually selected from the following: —H, linear and branched —CₙH₂ₙNH₂ wherein n=4, 5, 6, 7, 8, 9, 10, 11, 12, 14, 15, 16, 17, 18, 19, 20; —(O(CH₂CH₂))ₙOH wherein n=2, 3, 4, 5, 6; —(O(CH₂CH₂))ₙNH₂ wherein n=2, 3, 4, 5, 6; —CₙH₂ₙCOOH wherein n=3, 4, 5, 6, 7, 8, 9, 10, 11, 12, 14, 15, 16, 17, 18, 19; and/or

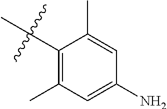 [SMILES: *c1c(C)cc(N)cc1C]

In further embodiments R³ may be each individually selected from the following: —H, —NH₂, —SH, —S—CₙH₂ₙ₊₁ wherein n=1-6, —OH, —Sn(CH₃)₃, Sn(C₄H₉)₃, —F, —Cl, —Br, —I, —B(OH)₂, —B(OCH₃)₂, —B(OiPr)₂, —B(pin), B(MIDA), -azide, -carbene. In certain embodiments R³ may be each individually selected from a heterocyclic ring fused to the GNR edge selected from the following:

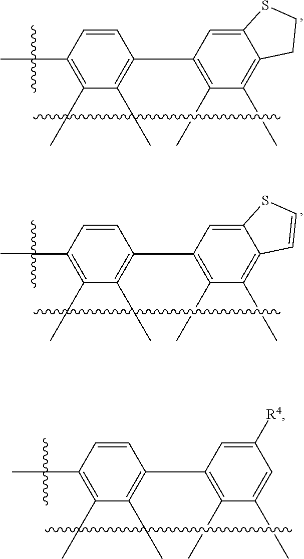

wherein R⁴ is selected from —NH₂, —SH, —S—CₙH₂ₙ₊₁ wherein n=1-6, —OH, —Sn(CH₃)₃, —Sn(C₄H₉)₃, F, —Cl, —Br, —I, —B(OH)₂, —B(OCH₃)₂, —B(OiPr)₂, —B(pin), —B(MIDA).

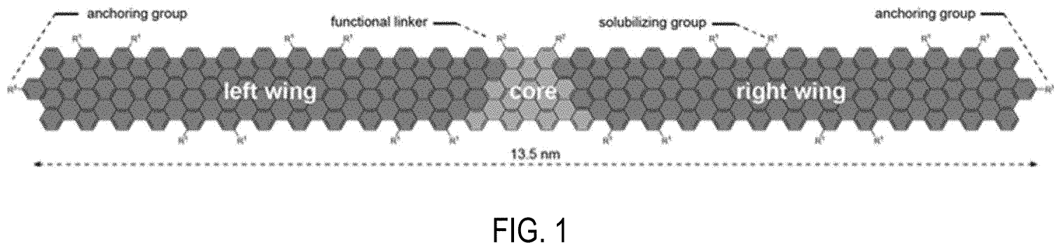

The present disclosure provides methods for making or synthesizing a precision GNR disclosed herein. In certain embodiments, the method for making or synthesizing a precision GNR disclosed herein may comprise any or all of the following steps: i) covalently coupling a metal electrode coordinating anchoring group to a first protected molecular precursor for GNRs using solution-based transition-metal-catalyzed cross-coupling reactions; ii) deprotection of the molecular precursor for GNRs followed by iterative transition-metal-catalyzed cross-coupling homologation with a second equivalent of the protected molecular precursor for GNRs extends the length of the GNR segment by one monomer unit; the process of deprotection and transition metal catalyzed cross-coupling homologation may be repeated until the polymer precursor reaches the desired length of the left/right wing of the GNR; iii) two equivalents of the wing may be covalently linked to opposite ends of a central functional GNR core using transition-metal-catalyzed cross-coupling reactions establishing the full length L of the ribbon; iv) oxidative cyclodehydrogenation either in solution or following deposition on catalytically active surfaces may give access to the fully conjugated graphitized GNR (see, e.g., FIG. 1). The iterative homologation may guarantee GNR samples featuring a monodisperse length distribution, precise widths, a regular pattern of solubilizing groups R¹, functional electrode anchoring groups R³ at either end of the ribbon, and a functional core at the center of the GNR.

Certain embodiments of the present disclosure may describe the design/engineering/synthesis of GNRs with suitable lengths to bridge a channel gap of X-20 nm between electrode contacts.

Certain embodiments of the present disclosure may describe the design/engineering/synthesis of GNRs comprising end-groups that selectively couple to and electrically conduct with metal (e.g. Au, Ag, Pd, Pt, Ru) contacts.

Certain embodiments of the present disclosure may describe the design/engineering/synthesis of GNRs featuring a precise band gap to maximize sensitivity.

Certain embodiments of the present disclosure may describe the design/engineering/synthesis of GNRs that are solution processable in organic and aqueous solvent mixtures.

Certain embodiments of the present disclosure may describe the design/engineering/synthesis of GNRs that are decorated with bioorthogonal linkers at one of more predetermined position along the ribbon that can be conjugated to a polymerase, a single stranded DNA or RNA oligo, an aptamer, an antibody, a protein, a small molecule, or a drug molecule.

FIG. 1 illustrates certain embodiments of a functional 9-AGNR with a discrete length featuring: i) solubilizing side chains (R¹) along the edges of the ribbon that increase the solubility in organic and/or aqueous processing solvents, ii) functional groups at the center of the ribbon that can be conjugated to molecular structures relevant to sensing (R²), and iii) anchoring groups (R³) that selectively bind to metal contacts at either end of the ribbon.

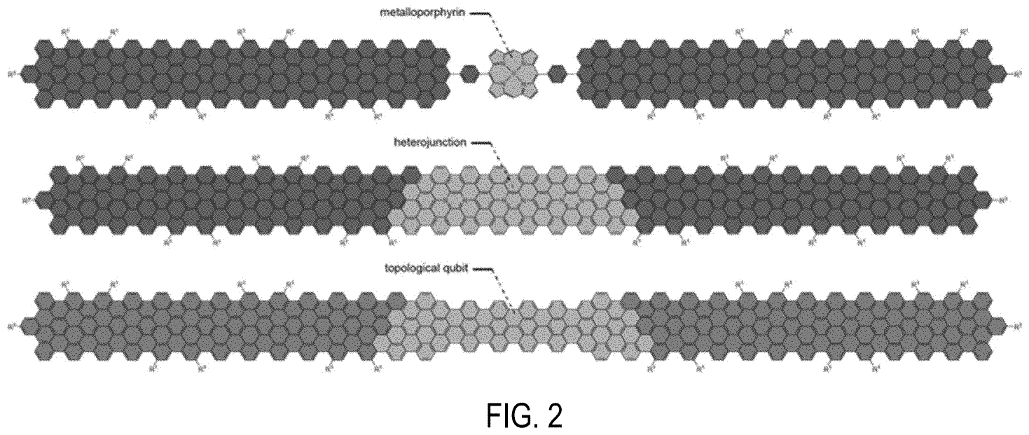

FIG. 2 illustrates certain embodiments of a functional 9-AGNR with a discrete length featuring: i) solubilizing side chains (R¹) along the edges of the ribbon that increase the solubility in organic and/or aqueous processing solvents, ii) a functional core at the center of the ribbon that features a metalloporphyrin, a p-n-p GNR heterojunction, or a double quantum dot qubit formed from two symmetry protected topological states, and iii) anchoring groups (R³) that selectively bind to metal contacts at either end of the ribbon.

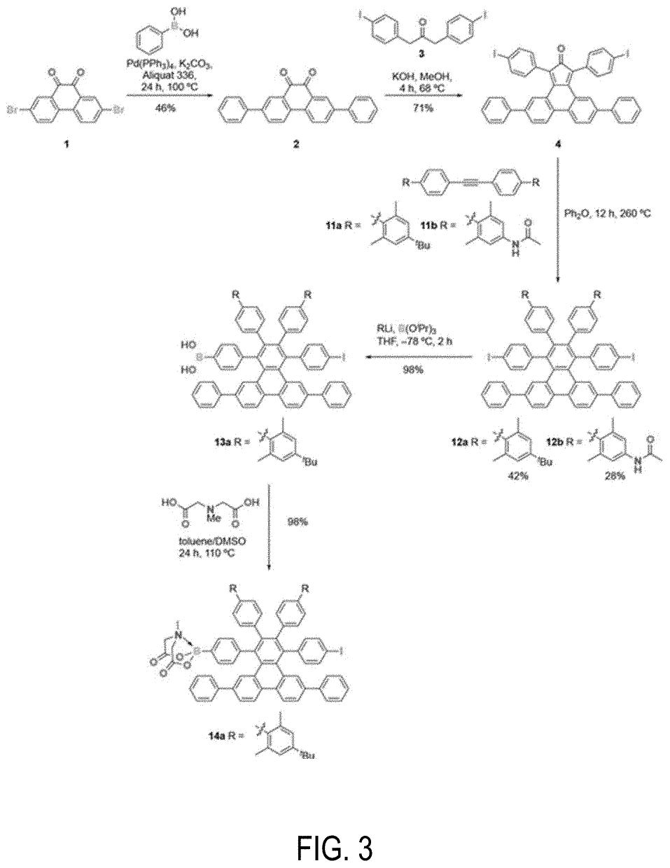

FIG. 3 illustrates an example of the bottom-up synthesis of a possible embodiment of a protected molecular building block 14a for a 9-AGNR.

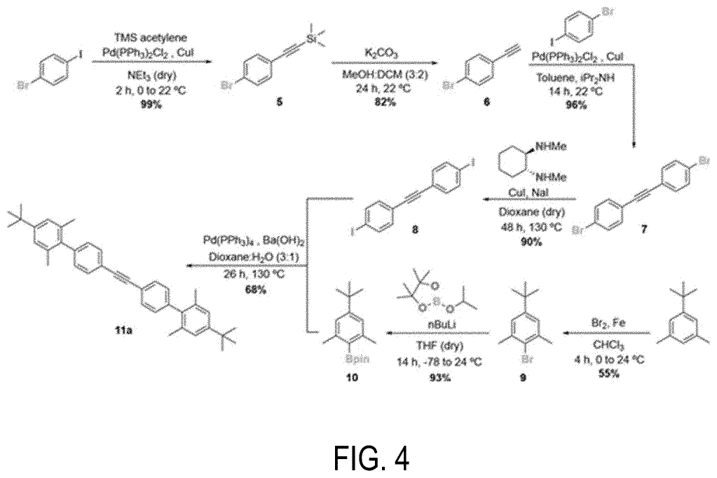

FIG. 4 illustrates an example of the bottom-up synthesis of 11a a key intermediate in the synthesis of molecular building block 14a featuring solubilizing groups compatible with organic solvents.

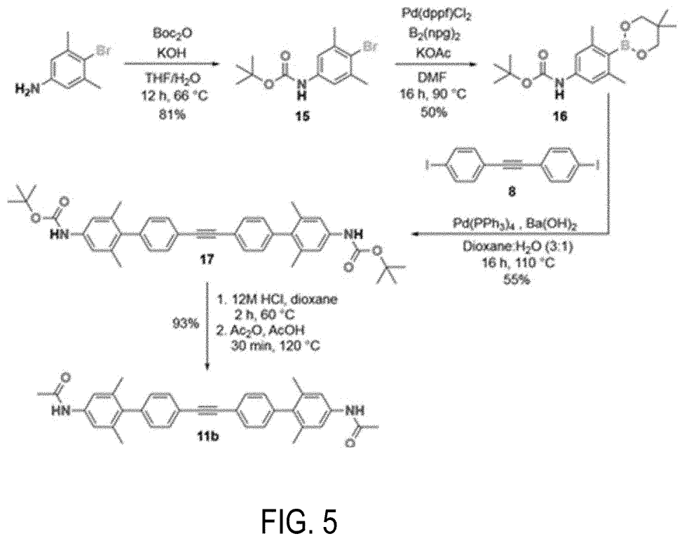

FIG. 5 illustrates an example of the bottom-up synthesis of 11b a key intermediate in the synthesis of molecular building block 12b representing a functional core.

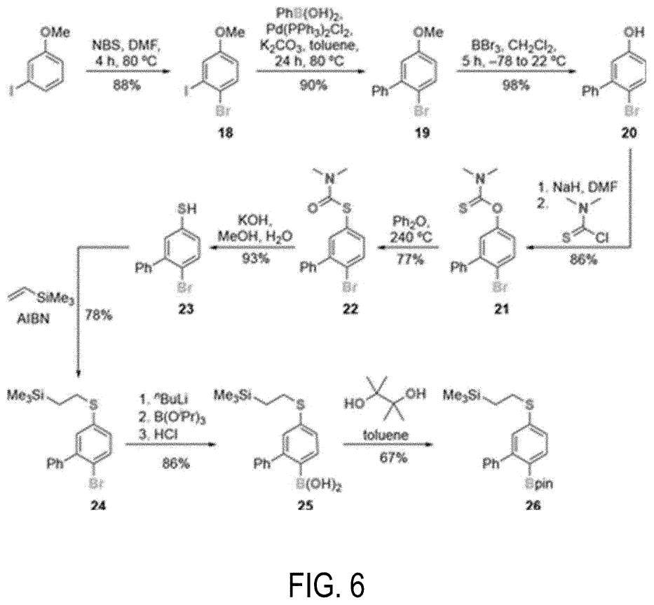

FIG. 6 illustrates an example of the bottom-up synthesis of a protected thiol-based anchoring group 26.

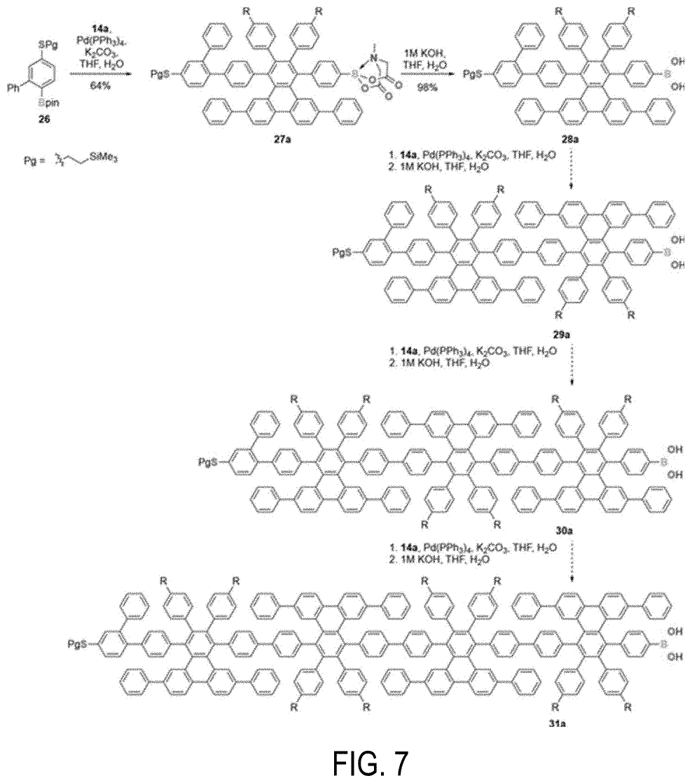

FIG. 7 illustrates an example of the functional integration of a protected thiol-based anchoring group along with the iterative cross-coupling homologation sequence that extends the length of the GNR polymer precursor by one monomer building block 14a at a time.

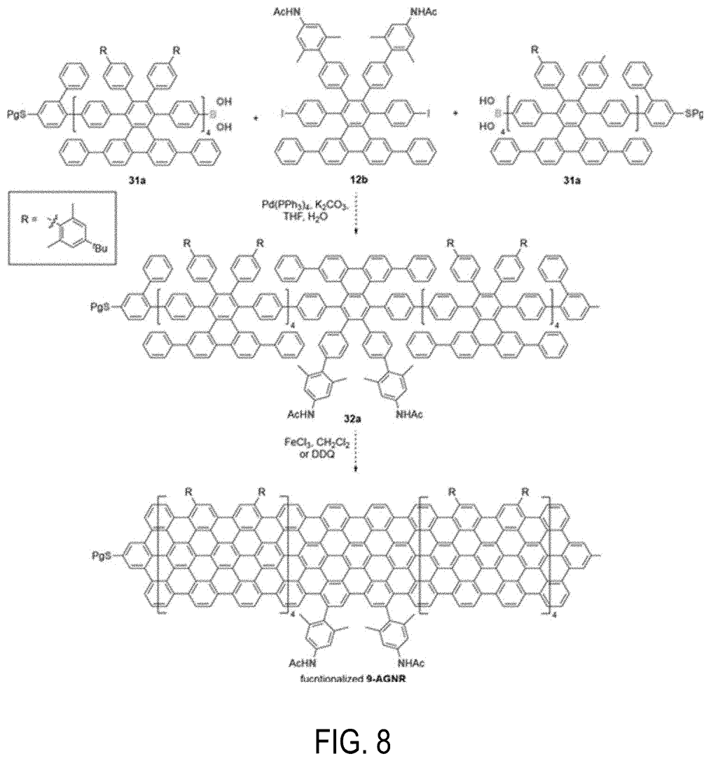

FIG. 8 illustrates an example of the final assembly of the left/right and right wing 31a with the functional core 12b to give a possible embodiment of a fully functionalized precision 9-AGNR.

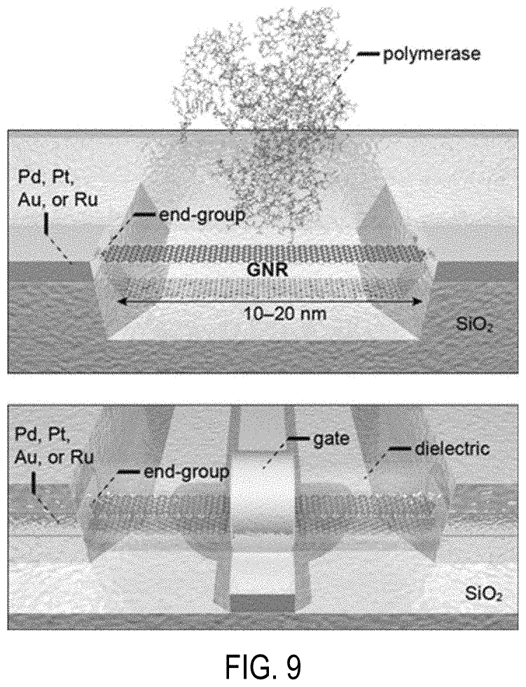

FIG. 9 illustrates certain embodiments of a GNR based sensor for DNA/RNA sequencing (top) and the possible embodiment of a GNR based electrical or magnetic switch for logic operations and computing.

In certain embodiments, a precision GNR bridge molecule can include any or all of the following: a central GNR with a precision structure, from among the following structural types: armchair, zigzag, cove, chevron, fjord, or a combination thereof; a functional anchoring groups at either end of the GNR from among the following types: amine, thiol, thioether, stannane, halide, boronic acid, boronic ester, azide, carbene; a central functional conjugation group at a precisely specified location, from among the following types: click; and edge group functionalization with solubilizing groups, from among the following types: linear and branched alkyl chains, substituted aromatic rings, oligoethylene glycol, carboxylic acids, sulfonic acids.

In certain embodiments, the molecule has a length of at least 10, 20, 30, 40, 50, or 100 or more fundamental repeat units of the GNR monomer building block.

In certain embodiments, the molecule has a width of N=5, 6, 7, 8, 9, 10, 11, 12, 13, 14, 15, 16, 17, 18, 19, 20; where N is the number of carbon atoms counted in a line across the width of the ribbon.

In certain embodiments, the molecule is fabricated by bottom-up synthetic chemical methods and bulk solution-phase synthesis, to achieve the precision structure, such as by the methods disclosed herein, and obvious variations on those disclosed methods.

In certain embodiments, a GNR molecular electronics nano-circuit can include any or all of the following: a single precision GNR bridge molecule of any of claims 1-4, having a length L, and end functionalization groups the conjugate to a select material; a pair of nano-electrodes have a gap of length <L, where the tips are made of said select material, or suitably coated or functionalized with said select material; and said GNR bridge coupled into said nano-gap, with end functional groups properly conjugated to the select material.

In certain embodiments, the nano-electrodes are coupled into a CMOS chip device.

Certain embodiments may include a GNR molecular electronics CMOS chip, comprising an array of pixels circuits, wherein each pixel circuit comprising a surface-exposed nano-electrode pair, and wherein each pixel nano-electrode pair provided with a GNR molecular electronic nano-circuit such as described above.

In certain embodiments, the GNR molecular bridges are assembled into place using voltage-driven trapping of molecules.

Certain embodiments may include a GNR molecular electronics sensor having any or all of the following: a GNR bridge such as described above; conjugated with a probe molecule at the precision internal conjugation site; and assembled into a circuit such as described above.

In certain embodiments, the probe molecule comprises a polymerase, a single stranded DNA or RNA oligo, an aptamer, an antibody, a protein, or a small molecule or drug molecule.

Certain embodiments may include a GNR molecular electronics sensor array chip, comprising a CMOS chip, with an array of pixel elements, each comprising a GNR molecular electronics sensor element such as described above.

In certain embodiments, a method of sequencing DNA, using a GNR molecular electronics sensor array chip, can include any or all of the following: proving a flow-cell that contains the chip and can provide liquid reagents; providing such a chip with polymerase-probe GNRs; supplying reagents in a flow cell that include sequencing reagents; recording signals from the sensors on the chip; processing the signals to basecalls; and recording and storing the called sequences.

In certain embodiments, a method can include any or all of the following: hybridization assay, aptamer binding assay, antibody binding assay, and molecular kinetics assay.

In certain embodiments, a precision GNR electrical or magnetic switch molecule can include any or all of the following: a central GNR with a precision structure, from among the following structural types: armchair, zigzag, cove, chevron, fjord, or a combination thereof; a functional anchoring groups at either end of the GNR from among the following types: amine, thiol, thioether, stannane, halide, boronic acid, boronic ester, azide, carbene; a central functional core at a precisely specified location, from among the following types: heterojunction interfaces, electron spin system, quantum dots, qubits, and/or symmetry protected topological states; and edge group functionalization with solubilizing groups, from among the following types: linear and branched alkyl chains, substituted aromatic rings, oligoethylene glycol, carboxylic acids, sulfonic acids.

In certain embodiments, the molecule has a length of at least 10, 20, 30, 40, 50, or 100 or more fundamental repeat units of the GNR monomer building block.

In certain embodiments, the molecule has a width of N=5, 6, 7, 8, 9, 10, 11, 12, 13, 14, 15, 16, 17, 18, 19, 20; where N is the number of carbon atoms counted in a line across the width of the ribbon.

In certain embodiments, the molecule is fabricated by bottom-up synthetic chemical methods and bulk solution-phase synthesis, to achieve the precision structure, such as by the methods disclosed herein, and obvious variations on those disclosed methods.

Certain embodiments can include a GNR molecular electronics nano-circuit having any or all of the following: a single precision GNR bridge molecule of any of claims 14-17, having a length L, and end functionalization groups the conjugate to a select material; a pair of nano-electrodes have a gap of length <L, where the tips are made of said select material, or suitably coated or functionalized with said select material; and said GNR electrical or magnetic switch coupled into said nanogap, with end functional groups properly conjugated to the select material.

In certain embodiments, the nano-electrodes are coupled into a CMOS chip device.

Certain embodiments can include a GNR molecular electronics CMOS chip, comprising an array of pixels circuits, wherein each pixel circuit comprising a surface-exposed nano-electrode pair and a gate, and wherein each pixel nano-electrode pair provided with a GNR molecular electronic nano-circuit such as described above.

In certain embodiments, the GNR electrical or magnetic switch is assembled into place using voltage-driven trapping of molecules.

In certain embodiments, the substituted aromatic rings include 2,4,6-trisubstituted aromatic rings.

In certain embodiments, the substituted aromatic rings include 2,4,6-trisubstituted aromatic rings.

As used herein and in the appended claims, the singular forms “a,” “an,” and “the” include plural referents unless the context clearly dictates otherwise. Thus, for example, reference to “a vector” includes a plurality of such vectors and reference to “the amino acid” includes reference to one or more amino acids and equivalents thereof known to those skilled in the art, and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although many methods and reagents are similar or equivalent to those described herein, the exemplary methods and materials are disclosed herein.

This written description uses examples to disclose the invention, including the best mode, and also to enable a person of ordinary skill in the relevant art to practice the invention, including making and using any devices or systems and performing any incorporated methods. The patentable scope of the invention is defined by the claims, and may include other examples that occur to those of ordinary skill in the art. Such other examples are intended to be within the scope of the claims if they have structural elements that do not differ from the literal language of the claims, or if they include equivalent structural elements with insubstantial differences from the literal languages of the claims.

1. A precision graphene nanoribbon (GNR) bridge molecule, comprising:
a central GNR having a precision structure selected the following structural types: armchair, zigzag, cove, chevron, and fjord;
a functional anchoring group at either end of the GNR selected from the following: amine, thiol, thioether, stannane, halide, boronic acid, boronic ester, azide, and carbene;
a central functional conjugation group at a precisely specified location; and
edge group functionalization with solubilizing groups selected from the following: linear and branched alkyl chains, substituted aromatic rings, oligoethylene glycol, carboxylic acids, and sulfonic acids.

2. The precision GNR bridge molecule of claim 1, where the molecule has a length of at least 10, 20, 30, 40, 50, or 100 or more fundamental repeat units of the GNR monomer building block.

3. The precision GNR bridge molecule of claim 1, where the molecule has a width of N=5, 6, 7, 8, 9, 10, 11, 12, 13, 14, 15, 16, 17, 18, 19, 20; where N is the number of carbon atoms counted in a line across the width of the ribbon.

4. The precision GNR bridge molecule of claim 1 where the molecule is fabricated by bottom-up synthetic chemical methods and bulk solution-phase synthesis.

5. The precision GNR bridge molecule of claim 1, wherein the substituted aromatic rings include 2,4,6-trisubstituted aromatic rings.

6. A GNR molecular electronics nano-circuit, comprising:
the precision GNR bridge molecule of claim 1, the precision GNR bridge molecule having a length L, where end functionalization groups the conjugate to a select material; and
a pair of nano-electrodes that have a gap of length <L, where tips are made of said select material or suitably coated or functionalized with said select material; where
the GNR bridge is coupled into a nano-gap, with end functional groups properly conjugated to the select material.

7. The GNR molecular electronics nano-circuit of claim 6, where the nano-electrodes are coupled into a CMOS chip device.

8. A GNR molecular electronics CMOS chip, comprising an array of pixels circuits, wherein each pixel circuit comprising a surface-exposed nano-electrode pair, and wherein each pixel nano-electrode pair provided with the GNR molecular electronics nano-circuit of claim 6.

9. The GNR molecular electronics CMOS chip of claim 8, where the GNR molecular bridges are assembled into place using voltage-driven trapping of molecules.

10. A GNR molecular electronics sensor, comprising:
the precision GNR bridge molecule of claim 1; and
conjugated with a probe molecule at the precision internal conjugation site.

11. The GNR molecular electronics sensor of claim 10, where the probe molecule comprises a polymerase, a single stranded DNA or RNA oligo, an aptamer, an antibody, a protein, or a small molecule or drug molecule.

12. A GNR molecular electronics sensor array chip, comprising a CMOS chip, with an array of pixel elements, each comprising the GNR molecular electronics sensor of claim 10.

13. A method of sequencing DNA, using a graphene nanoribbon (GNR) molecular electronics sensor array chip, the method comprising:
proving a flow-cell that contains the chip and can provide liquid reagents;
providing such a chip with polymerase-probe GNRs;
supplying reagents in a flow cell that include sequencing reagents;
recording signals from the sensors on the chip;
processing the signals to basecalls; and
recording and storing the called sequences.

14. A precision graphene nanoribbon (GNR) switch molecule, comprising:
a central GNR with a precision structure selected from the following structural types: armchair, zigzag, cove, chevron, and fjord;
a functional anchoring group at either end of the GNR selected from the following: amine, thiol, thioether, stannane, halide, boronic acid, boronic ester, azide, and carbene;
a central functional core at a precisely specified location selected from among the following types: heterojunction interfaces, electron spin system, quantum dots, qubits, and/or symmetry protected topological states; and
edge group functionalization with solubilizing groups, from among the following types: linear and branched alkyl chains, substituted aromatic rings, oligoethylene glycol, carboxylic acids, sulfonic acids.

15. The precision GNR switch molecule of claim 14, where the molecule has a length of at least 10, 20, 30, 40, 50, or 100 or more fundamental repeat units of the GNR monomer building block.

16. The precision GNR switch molecule of claim 14, where the molecule has a width of N=5, 6, 7, 8, 9, 10, 11, 12, 13, 14, 15, 16, 17, 18, 19, 20; where N is the number of carbon atoms counted in a line across the width of the ribbon.

17. The precision GNR switch molecule of claim 14 where the molecule is fabricated by bottom-up synthetic chemical methods and bulk solution-phase synthesis.

18. A GNR molecular electronics nano-circuit, comprising:
the precision GNR switch molecule of claim 1 having a length L, where end functionalization groups the conjugate to a select material; and
a pair of nano-electrodes that have a gap of length <L, where tips are made of said select material, or suitably coated or functionalized with said select material; where
said GNR electrical or magnetic switch coupled into a nanogap, with end functional groups properly conjugated to the select material.

19. The GNR molecular electronics nano-circuit of claim 18, where the nano-electrodes are coupled into a CMOS chip device.

20. A GNR molecular electronics CMOS chip, comprising an array of pixels circuits, wherein each pixel circuit comprising a surface-exposed nano-electrode pair and a gate, and wherein each pixel nano-electrode pair provided with the GNR molecular electronics nano-circuit of claim 18.
